(19) United States
(12) Patent Application Publication
(10) Pub. No.: US 2008/0036999 A1
(43) Pub. Date: Feb. 14, 2008

(54) SPECTROSCOPIC EXAMINATION OF BODY FLUID AND TISSUE SAMPLES TO AID DIAGNOSIS OF ALZHEIMER'S DISEASE

(76) Inventors: Wolfgang Petrich, Bad Schonborn (DE); Joachim Moecks, Mannheim (DE)

Correspondence Address: ROCHE DIAGNOSTICS OPERATIONS INC., 9115 Hague Road, Indianapolis, IN, 46250-0457, US

(21) Appl. No.: 11/762,978

(22) Filed: Jun. 14, 2007

Related U.S. Application Data
(63) Continuation of application No. PCT/EP05/12151, filed on Nov. 12, 2005.

(30) Foreign Application Priority Data
Dec. 18, 2004 (DE) 102004061064.9

(51) Int. Cl. G01N 33/48; G01J 3/00

(52) U.S. Cl. 356/39; 356/51

(57) ABSTRACT

A method is described for the spectroscopic examination of samples which are taken from human tissue or body fluids, in particular blood, comprising the following steps: measuring an infrared spectrum of a sample to be examined; analysing the measured infrared spectrum in order to determine at least one sample parameter that characterizes the infrared spectrum in at least one of the following wavenumber ranges 2850 cm⁻¹ to 2886 cm⁻¹, 2798 cm⁻¹ to 2835 cm⁻¹, 1723 cm⁻¹ to 1733 cm⁻¹, 1429 cm⁻¹ to 1465 cm⁻¹, 1338 cm⁻¹ to 1364 cm⁻¹, 1089 cm⁻¹ to 1123 cm⁻¹, 1034 cm⁻¹ to 1074 cm⁻¹, comparing the at least one sample parameter with at least one reference parameter that was obtained by analysing at least one reference spectrum that was measured on at least one sample of body fluid from patients afflicted by Alzheimer's, and assigning the sample to a class with an increased suspicion of Alzheimer's disease if the deviation of the at least one sample parameter from the at least one reference parameter does not exceed a predetermined threshold value.

### RELATED APPLICATIONS

This application is a continuation of PCT application PCT/EP2005/012151 filed Nov. 12, 2005 and claims priority to German application DE 102004061064.9 filed Dec. 18, 2004.

### FIELD OF THE INVENTION

The invention concerns a method and a device for spectroscopically examining human tissue samples and body fluids, in particular blood, with regard to an increased suspicion of Alzheimer's disease.

### BACKGROUND

Alzheimer's is a widespread disease which is extraordinarily difficult to diagnose especially in its early stages. About 6% to 8% of all people over 65 years of age have Alzheimer's. Initially the symptoms of this disease are relatively mild and are often misinterpreted as common symptoms of old age. As the disease progresses, the symptoms escalate from mild forgetfulness to a sever disturbance of all cognitive functions of the brain. However, a definitive diagnosis of Alzheimer's disease can only be currently made post mortem by means of a histopathological examination of brain tissue.

### SUMMARY OF THE INVENTION

The object of the invention is therefore to identify a method which can make it easier for a doctor to make an early diagnosis of Alzheimer's disease.

This object is achieved by a method for the spectroscopic examination of samples which are taken from human tissue or body fluids, in particular blood, comprising the following steps:
measuring an infrared spectrum of a sample of the body fluid to be examined;
analysing the measured infrared spectrum in order to determine at least one sample parameter that characterizes the infrared spectrum in at least one, preferable at least two, particularly preferably at least three and in particular at least four of the following wavenumber ranges:
   2850 cm⁻¹ to 2886 cm⁻¹,
   2798 cm⁻¹ to 2835 cm⁻¹,
   1723 cm⁻¹ to 1733 cm⁻¹,
   1429 cm⁻¹ to 1465 cm⁻¹,
   1338 cm⁻¹ to 1364 cm⁻¹,
   1089 cm⁻¹ to 1123 cm⁻¹, and
   1034 cm⁻¹ to 1074 cm⁻¹;
comparing the at least one sample parameter with at least one reference parameter that was obtained by analysing at least one reference spectrum that was measured on at least one sample of body fluid from patients afflicted by Alzheimer's; and
assigning the sample to a class with an increased suspicion of Alzheimer's disease if the deviation of the at least one sample parameter from the at least one reference parameter does not exceed a predetermined threshold value.

The object is additionally achieved by a device for examining samples that are taken from human tissue or body fluids, in particular blood, comprising:
a light source for emitting infrared light;
a spectrometer for measuring an infrared spectrum of a sample of the body fluid to be examined;
an analytical unit which, when in operation
   determines at least one sample parameter by analysing the measured infrared spectrum where the sample parameter characterizes the infrared spectrum in at least one of the following wavenumber ranges:
      2850 cm⁻¹ to 2886 cm⁻¹,
      2798 cm⁻¹ to 2835 cm⁻¹,
      1723 cm⁻¹ to 1733 cm⁻¹,
      1429 cm⁻¹ to 1465 cm⁻¹,
      1338 cm⁻¹ to 1364 cm⁻¹,
      1089 cm⁻¹ to 1123 cm⁻¹, and
      1034 cm⁻¹ to 1074 cm⁻¹;
   compares the at least one sample parameter with at least one reference parameter that was obtained by analysing at least one reference spectrum which was measured on at least one sample of body fluid from patients afflicted by Alzheimer's disease; and
   assigns the sample to a class with an increased suspicion of Alzheimer's disease if the deviation of the at least one parameter from the at least one reference parameter does not exceed a predetermined threshold value.

### BRIEF DESCRIPTION OF THE DRAWING

FIG. 1 shows an absorption spectrum of a blood sample from an Alzheimer's patient.

### DETAILED DESCRIPTION OF THE INVENTION

In a difference context, a method for analysing clinically relevant liquids with regard to hepatic cirrhosis or diabetes is known from EP 0 644 412 A2. The known method comprises the following steps:
a) measuring infrared spectra of a plurality of samples which belong to known classes i.e. which originate from patients with diabetes or hepatic cirrhosis or healthy reference persons,
b) carrying out a multivariate analytical procedure while minimizing errors in the assignment of samples to the known classes,
c) storing the parameters of the multivariate analytical procedure obtained by minimization,
d) providing a sample to be examined,
e) applying the sample to a carrier,
f) drying the sample,
g) irradiating the sample with infrared radiation,
h) recording an infrared spectrum of the sample,
i) analysing the infrared spectrum by means of a multivariate analytical procedure, and
j) outputting the assignment of the sample to a class of the multivariate analytical procedure.

The article by W. Petrich in Applied Spectroscopy Reviews, 36 (2 & 3), 181-237 (2001) provides an overview of the use of infrared spectroscopic examinations in the field of medical diagnostics.

Within the scope of the present invention it was found that the method known from EP 0 644 412 A2 can also aid in the diagnosis of Alzheimer's disease in that samples can be assigned to a class with an increased suspicion of Alzheimer's disease. With regard to the measuring technology and multivariate analytical procedures that can be advantageously applied, reference is made to EP 0 644 412 A2 the disclosure of which is herein incorporated by reference.

In particular it was found within the scope of the invention that a sample can be assigned to a class with an increased suspicion of Alzheimer's disease with surprising reliability by determining a sample parameter that characterizes the infrared spectrum in at least one of the above-mentioned wavenumber ranges.

This sample parameter is preferably obtained by a multivariate analytical procedure for example by discriminant analysis, cluster analysis or neuronal networks. The informative value of the sample parameter is higher if several of the above-mentioned wavenumber ranges and in particular the ranges from 1034 cm⁻¹ to 1074 cm⁻¹, 1089 cm⁻¹ to 1123 cm⁻¹ and/or 2798 cm⁻¹ to 2835 cm⁻¹ are included in the analysis such that the sample parameter characterizes the infrared spectrum in several wavenumber ranges.

In an analysis, a sample spectrum can be represented by one point in a multidimensional parameter space. A reference spectrum can be represented in the same manner in the parameter space as a point, and a group of reference spectra can be represented as a scatter plot. The coordinates of the points characterizing the sample or reference spectra can be interpreted as sample or reference parameters. By way of illustration the assignment of the sample to a class having an increased suspicion of Alzheimer's disease can be made based on the distance between points representing the sample and reference spectra. However, it is not necessary to determine the distance in terms of a numerical value. The assignment can for example also be made by means of a neuronal network and it is merely determined that the corresponding spectra are sufficiently similar in the spectral range(s) that are important for the invention to allow an assignment to be made.

An increased suspicion of Alzheimer's disease can be determined particularly reliably if the threshold value for the deviation of the sample parameter from the reference parameter is not given as an absolute value, but rather depends on the result of a comparison of the sample parameter with a second reference parameter. Just as the first reference parameter characterizes one or more spectra which were measured on samples from patients with Alzheimer's disease, the second reference parameter characterizes spectra which were measured on samples of persons of a reference group. This reference group can for example be healthy persons. Another possibility is that the persons of the reference group suffer from a dementia that is different from Alzheimer's disease such as vascular dementia. The symptoms of different types of dementia are often very similar such that diagnosis may be difficult even for experienced doctors. It is often also difficult to differentiate between Alzheimer's disease and other perception or memory disorders and mental diseases, in particular depressions. Diseases of this type are summarized in the English speaking literature by the term cognitive impairment. Even in such difficult cases, the application of the method according to the invention can make it much easier for the doctor to make a correct diagnosis.

Further details and advantages of the invention are elucidated on the basis of an embodiment example with reference to the attached drawing. The special features illustrated therein can be used individually or in combination in order to create preferred embodiments of the invention.

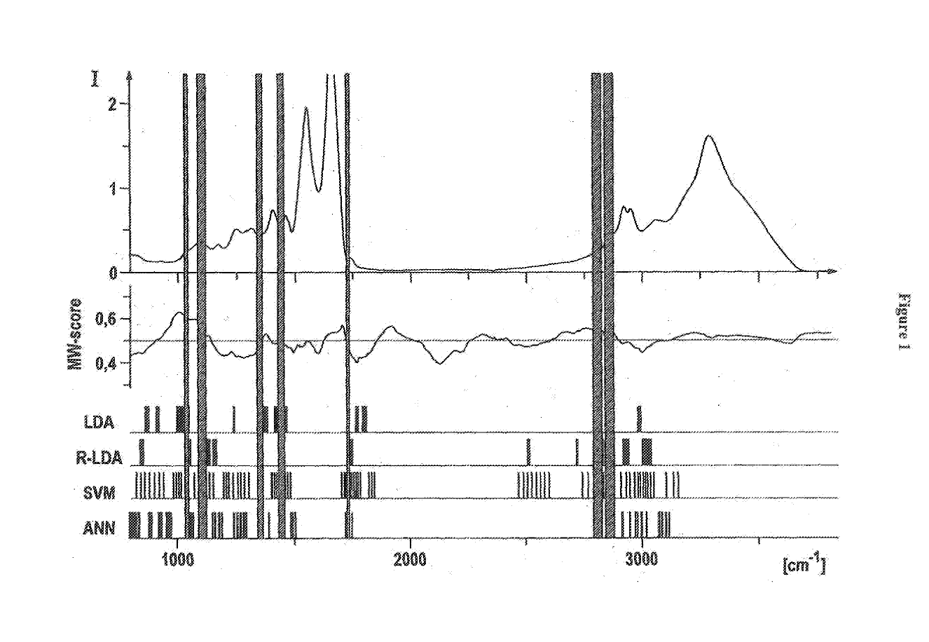

FIG. 1 shows an absorption spectrum of a blood sample from an Alzheimer's patient. The upper third of FIG. 1 shows a typical absorption spectrum that was measured on a dried sample of blood serum from a patient with Alzheimer's disease. The intensity is plotted in arbitrary units against the wavenumber in cm⁻¹. Wavenumber ranges that are particularly important for assigning the sample to a class having an increased suspicion of Alzheimer's disease are shown in FIG. 1 by continuous bars. These particularly important wavenumber ranges are: 1034 cm⁻¹ to 1074 cm⁻¹, 1089 cm⁻¹ to 1123 cm⁻¹, 1338 cm⁻¹ to 1364 cm⁻¹, 1429 cm⁻¹ to 1475 cm⁻¹, 1723 cm⁻¹ to 1733 cm⁻¹, 2798 cm⁻¹ to 2835 cm⁻1 and 2850 cm⁻¹ to 2886 cm⁻¹.

A sample parameter was determined by using a multivariate analytical procedure, for example linear discriminant analysis or neuronal networks which characterizes the sample spectrum shown in the said wavenumber ranges. In order to decide whether this sample is to be assigned to a class having an increased suspicion of Alzheimer's disease, the sample parameter is compared with a reference parameter. The reference parameter characterizes reference spectra in the said wavenumber ranges which were measured on samples of body fluids from patients with Alzheimer's disease. Both the reference parameter and the sample parameter are determined by using a multivariate analytical procedure.

If several reference spectra are used—which is preferred—it is possible to firstly determine a reference parameter for each reference spectrum separately and then compare the sample parameter with a value derived from the individual reference parameters e.g. the mean. Another method is to determine a characteristic general reference spectrum for the said wavenumber ranges from several measured reference spectra and then determine a single reference parameter for this spectrum for the comparison with the sample parameter.

If the deviation of the at least one sample parameter from the at least one reference parameter is below a predetermined threshold value, the sample is assigned to a class having an increased suspicion of Alzheimer's disease.

The Mann-Whitney Score (MW-Score) is plotted below the absorption spectrum in FIG. 1 for the respective wavenumber of the spectrum. The Mann-Whitney score is a measure of the reliability of an assignment of the sample to a class having an increased suspicion of Alzheimer's disease that is made exclusively on the basis of the respective wavenumber. In other words the Mann-Whitney score indicates bow characteristic the intensity of a spectrum at a certain wavenumber is for an Alzheimer's disease. It is evident that the Mann-Whitney score does not show any pronounced maxima and thus a reliable assignment of a sample spectrum to a class having an increased suspicion of Alzheimer's disease appears at first site to be futile. It is therefore even more surprising that such an assignment can nevertheless be made on the basis of the marked wavenumber ranges with a reliability that is sufficiently high to provide a valuable decision aid to a physician in the diagnosis of Alzheimer's disease. In this context, it is not even obligatory to take into consideration all six stated wavenumber ranges for determining the sample parameter.

In the lower third of FIG. 1 black bars mark wavenumber ranges that have proven to be indicative of Alzheimer's disease when using the multivariate analytical procedure that is in each case indicated by its abbreviation.

The multivariate analytical procedures that were used were linear discriminant analysis LDA, robust linear discriminant analysis R-LDA, support vector machines SVM and neuronal networks (artificial neural network) ANN. It can be seen that there are considerable differences between the ranges identified as being characteristic by the different multivariate analytical procedures which also seems to make a reliable assignment appear hopeless at first. However, upon closer inspection it is evident that for some wavenumber ranges there is agreement between the above-mentioned four multivariate analytical procedures in finding an increased significance for the presence of Alzheimer's disease. These ranges are highlighted in FIG. 1 by continuous bars and are used in the described method to assign a sample or an infrared spectrum measured thereon to a class with an increased suspicion of Alzheimer's disease.

What is claimed is:

1. A method for aiding a diagnosis of Alzheimer's disease in a patient comprising the steps of:
providing a body fluid or tissue sample from the patient,
measuring an infrared spectrum of said sample,
analysing the measured infrared spectrum and determining a sample parameter that characterizes the infrared spectrum in a wavenumber range selected from the group consisting of 2850 cm⁻¹ to 2886 cm⁻¹, 2798 cm⁻¹ to 2835 cm⁻¹, 1723 cm⁻¹ to 1733 cm⁻¹, 1429 cm⁻¹ to 1465 cm⁻¹, 1338 cm⁻¹ to 1364 cm⁻¹, 1089 cm⁻¹ to 1123 cm⁻¹, and 1034 cm⁻¹ to 1074 cm⁻¹,
comparing the sample parameter with a reference parameter obtained by analyzing a reference spectrum measured on a body fluid or tissue reference sample from a reference patient afflicted with Alzheimer's disease, and
assigning the patient to a class with an increased suspicion of Alzheimer's disease if the deviation of the patient's sample parameter from the reference parameter does not exceed a predetermined threshold value.

2. The method of claim 1 wherein the predetermined threshold value depends on the result of a comparison of the sample parameter with a second reference parameter obtained by analyzing a second reference spectrum measured on a body fluid or tissue sample from a reference group of patients.

3. The method of claim 2 wherein the reference group is healthy.

4. The method of claim 2 wherein the reference group suffers from vascular dementia.

5. The method of claim 2 wherein the reference group suffers from a perception or memory disorder other than Alzheimer's disease.

6. The method of claim 2 wherein the reference group suffers from depression.

7. The method of claim 1 wherein the sample and reference parameters are determined using a multivariate analytical procedure.

8. The method of claim 1 wherein the sample is a blood sample.

9. The method of claim 1 wherein the sample is dried before measurement.

10. The method of claim 1 wherein the infrared spectrum of the sample is measured with a Fourier spectrometer.

11. A device for examining a body fluid or tissue sample from a patient in order to aid a diagnosis of Alzheimer's disease, the device comprising:
a light source for emitting infrared light,
a spectrometer for measuring an infrared spectrum from said sample, and
an analytical unit which, when in operation,
determines a sample parameter by analyzing the measured infrared spectrum where the sample parameter characterizes the infrared spectrum in a wavenumber range selected from the group consisting of 2850 cm⁻¹ to 2886 cm⁻¹, 2798 cm⁻¹ to 2835 cm⁻¹, 1723 cm⁻¹ to 1733 cm⁻¹, 1429 cm⁻¹ to 1465 cm⁻¹, 1338 cm⁻¹ to 1364 cm⁻¹, 1089 cm⁻¹ to 1123 cm⁻¹, and 1034 cm⁻¹ to 1074 cm⁻¹,
compares the sample parameter with a reference parameter obtained by analyzing a reference spectrum measured on a body fluid or tissue sample from a patient afflicted with Alzheimer's disease, and
assigns the patient to a class having an increased suspicion of Alzheimer's disease if the deviation of the parameter from the reference parameter does not exceed a predetermined threshold value.
